(19) United States
(12) Patent Application Publication
(10) Pub. No.: US 2008/0167595 A1
(43) Pub. Date: Jul. 10, 2008

(54) METHOD FOR CONNECTING IMPLANTED CONDUITS

(75) Inventors: Christopher H. Porter, Woodinville, WA (US); Tuan Doan, Burnsville, MN (US); Laurie E. Lynch, Eden Prairie, MN (US)

Correspondence Address: KNOBBE MARTENS OLSON & BEAR LLP, 2040 MAIN STREET, FOURTEENTH FLOOR, IRVINE, CA, 92614, US

(73) Assignee: GRAFTcath Inc., Eden Prairie, MN (US)

(21) Appl. No.: 12/054,128

(22) Filed: Mar. 24, 2008

Related U.S. Application Data
(62) Division of application No. 10/962,200, filed on Oct. 8, 2004.
(60) Provisional application No. 60/509,428, filed on Oct. 8, 2003.
(60) Provisional application No. 60/605,681, filed on Aug. 31, 2004.

(51) Int. Cl. A61M 39/00

(52) U.S. Cl. 604/8

(57) ABSTRACT

Methods and devices are disclosed for connecting implantable body fluid conduits, such as catheters and grafts for AV shunts. A connector with thin connector walls at the lumen openings provides a connecting lumen that is close to flush with the lumens of the attached conduits. A tapered, smooth walled connector lumen allows connection of conduits with different internal diameters while preserving laminar flow in the transition between different conduit diameters. Rounding of the connector edges at the lumen openings further reduce disturbances in flow.

### CROSS-REFERENCE TO RELATED APPLICATIONS

This application is a divisional application of U.S. patent application Ser. No. 10/962,200 filed on Oct. 8, 2004, which claims the benefit of U.S. Provisional Application No. 60/509,428 filed on Oct. 8, 2003 under 35 U.S.C. §119(e), and which also claims the benefit of U.S. Provisional Application No. 60/605,681 filed on Aug. 31, 2004, the disclosures of both of which are incorporated by reference herein in their entirety.

### BACKGROUND OF THE INVENTION

1. Field of the Invention

This invention relates generally to connectors used to join fluid conduits within the body. These fluid conduits may include AV grafts, implantable catheters, left ventricular assist devices, as well as native tissue vessels.

2. Description of the Related Art

In the United States, approximately 300,000 people have end-stage renal disease requiring chronic hemodialysis. Although many materials that have been used to create prosthetic arterio-venous (AV) grafts have also been tried for dialysis access, expanded polytetrafluoroethylene (ePTFE) has become the material of choice. The reasons for this include its ease of needle puncture and particularly low complication rates (pseudo-aneurysm, infection, and thrombosis). However, patency rates of ePTFE access grafts are still not satisfactory and overall graft failure rates remains high. Sixty percent of these grafts fail yearly, usually due to stenosis at the venous end. (See Besarab, A & Samararpungavan D., “Measuring the Adequacy of Hemodialysis Access”. Curr Opin Nephrol Hypertens 5(6) 527-531, 1996, Raju, S. “PTFE Grafts for Hemodialysis Access”. Ann Surg 206(5), 666-673, November 1987, Koo Seen Lin, L C & Burnapp, L. “Contemporary Vascular Access Surgery for Chronic Hemodialysis”. J R Coll Surg 41, 164-169, 1996, and Kumpe, D A & Cohen, M A H “Angioplasty/Thrombolytic Treatment of Failing and Failed Hemodialysis Access Sites: Comparison with Surgical Treatment”. Prog Cardiovasc Dis 34(4), 263-278, 1992, all herein incorporated by reference in their entirety.) These failure rates are increased in higher-risk patients, such as diabetics. These access failures result in disruption of routine dialysis schedules and create hospital costs of over $2 billion per year. (See Sharafuddin, M J A, Kadir, S., et al. “Percutaneous Balloon-assisted aspiration thrombectomy of clotted Hemodialysis access Grafts”. J Vasc Interv Radiol 7(2) 177-183, 1996, herein incorporated by reference in its entirety).

### SUMMARY OF THE INVENTION

In one embodiment, a biocompatible connector for joining body fluid conduits is provided. The connector comprises an elongate body, the elongate body comprising a first end having a first outer diameter and adapted to receive a first body fluid conduit, a second end adapted to receive a second body fluid conduit, and a lumen between the first end and the second end of the elongate body, the lumen comprising an first opening and an second opening, and a length, a first edge about the first opening, and a second edge about the second opening, wherein the first opening diameter is at least about 90% of the first outer diameter. In further embodiments, the first opening diameter may at least about 95% of the first outer diameter, sometimes at least about 98% of the first outer diameter. The first opening may be an inflow opening or an outflow opening. The second opening may be an inflow opening or an outflow opening. The first edge may comprise a smoothed surface. The elongate body may further comprise a first transition zone within the lumen, the first transition zone comprising a first inner diameter and a second inner diameter located generally between the first opening and the second opening, wherein the first inner diameter is greater than the second inner diameter. The elongate body may also comprise a second transition zone within the lumen, the second transition zone comprising a third inner diameter and a fourth inner diameter located generally between the second inner diameter and the second opening. In one embodiment, the third inner diameter is greater than the fourth inner diameter. In another embodiment, the third inner diameter is less than the fourth inner diameter. The change in diameter from the first inner diameter to the second inner diameter may be linear. The first inner diameter may be located about the first opening. The second inner diameter may be located at a distance of at least about 20% of the lumen length from the first inner diameter, sometimes at least about 50% of the lumen length from the first inner diameter, and occasionally no greater than about 90% of the lumen length from the first inner diameter. The lumen wall in the first transition zone may form an angle of less than about 20 degrees with respect to the longitudinal axis of the lumen, sometimes less than about 10 degrees, and preferably less than about 5 degrees. Occasionally, the lumen wall in the first transition zone forms an angle of less than about 3 degrees with respect to the longitudinal axis of the lumen. The elongate body may further comprise a middle segment between the first end and the second end. The middle segment may comprise a central flange. The middle segment may also comprise a first indentation region. The elongate body may comprise a material selected from the group comprising titanium or a titanium alloy, nickel or a nickel alloy, MP35N, stainless steel, polysuflone, PEEK, nylon, polypropylene or polyethylene or any flexible or chip-resistant polymer. The biocompatible connector may further comprise a first securing device capable of exerting a radially inward force against the first conduit at the first indentation region. The first securing device may comprise a suture, a twisted wire, a tension clip, a crimp ring, a clamshell assembly, a collet assembly, or a compression sleeve. The middle segment may also comprise a mechanical interlock interface capable of joining and separating the first end and the second end of the elongate body. The middle segment may also comprise a lumen access interface. The lumen access interface may be adapted for leak-resistant needle puncture access. The lumen access interface may be subcutaneous or transcutaneous. The biocompatible connector may further comprise a connector sleeve with a first end and a second end, and a sleeve lumen therebetween, the tubular sleeve having a first expanded sleeve configuration and a second reduced sleeve configuration, the second reduce sleeve configuration capable of exerting a radially inward bias. The compression sleeve may comprise a material selected from the group comprising silicone, polyurethane, spring metal, a flexible polymer and a chip-resistant polymer. The biocompatible connector may further comprise a strain relief assembly positioned about the first end of the elongate body. The strain relief assembly may comprise a wire or polymer coil. The elongate body may have a first wall thickness measured in the lumen at a distance of no greater than about 1 mm from the first opening, sometimes at a distance of no greater than about 0.5 mm from the first opening and occasionally at the inflection point between the first edge and the lumen. The first wall thickness may be generally within the range of about 0.030 mm to about 0.250 mm, about 0.075 mm to about 0.200 mm or about 0.100 mm to about 0.180 mm.

In another embodiment, a system for treating renal disease is provided. The system comprises a graft having a first end configured for anastomosis to a blood vessel, a second end adapted to connect to a catheter, and a lumen between the first end and the second end, and a catheter having a first end configured for insertion into a vein, a second end adapted to connect to a graft, and a lumen between the first end and the second end, wherein the second end of the catheter has a wall thickness at a measuring point defined at the inflection point between the first edge and the lumen, and the wall thickness is no greater than about 0.250 mm. The wall thickness may be within the range of about 0.030 mm to about 0.250 mm, about 0.075 mm to about 0.200 mm, or about 0.100 mm to about 0.180 mm.

In another embodiment of the invention, a method for implanting a body fluid conduit is provided, comprising the steps of providing a first body fluid conduit, a second body fluid conduit and a connector having a first end, second end, a lumen between the first end and second end, and a first wall thickness at the first end, wherein the first wall thickness is no greater than about 0.250 mm, attaching the first body fluid conduit to a blood vessel, inserting the second body fluid conduit into a blood vessel, connecting the first body fluid conduit to the first end of the connector, and connecting the second body fluid conduit to the second end of the connector. In some embodiments, the step of connecting the first body fluid conduit to the second end of the connector is performed before the inserting step. In some embodiments, the step of connecting the second body fluid conduit to the second end of the connector is performed before the inserting step. In some embodiments, the first end and/or second end of the connector may be preattached to the first body fluid conduit and/or second body fluid conduit, respectively.

In another embodiment, a hemodialysis and vascular access system is provided, comprising an indwelling tubular conduit having a first section provided from a material which is biocompatible with and adapted for attachment to an artery and a second section adapted to be inserted within a vein at an insertion site, said second section having an outside diameter which is less than an inner diameter of the vein at the insertion site and having at least one opening in an end thereof which is distant from the insertion site such that, in operation, blood flows from the artery through the conduit and is returned to the vein through the at least one opening and blood also flows through the vein uninterrupted around the outside of the second section, and a connector connecting the first and the second sections, the connector having a tubular body with a central lumen extending therethrough, wherein the central lumen has a first inside diameter adjacent a transition to the first section and a second inside diameter adjacent a transition to the second section, and a nonturbulent transition in the lumen between the first diameter and the second diameter. The first section may comprise ePTFE, polyurethane, silicone or Dacron®. The first section may have an inside diameter within the range of from about 5.5 mm to about 6.5 mm, and sometimes about 5 mm to about 7 mm. The second section may comprise a silastic material or silicone. A downstream end of the second section may be provided with a bevel. The hemodialysis and vascular access system may additionally comprise an access segment for receiving a needle to allow access to blood flowing through the conduit. The first inside diameter is at least about 95% of a corresponding outside diameter, or even at least about 98% of a corresponding outside diameter. In some embodiments, at least one edge about an opening of the central lumen comprises a smoothed surface. The nonturbulent transition in the lumen may be linear. The second inside diameter may be located at a distance of at least about 20% of the lumen length from the first inside diameter, sometimes at least about 50% of the lumen length from the first inside diameter and occasionally no greater than about 90% of the lumen length from the first inside diameter. The lumen wall about the nonturbulent transition may be angled less than about 20 degrees with respect to the longitudinal axis of the lumen, preferably less than about 10 degrees or 5 degrees, and occasionally less than about 3 degrees with respect to the longitudinal axis of the lumen. The connector may comprise a middle segment between the first section and the second section. The middle segment may comprise a central flange. The middle segment may also comprise a first indentation region. The connector may comprise a material selected from the group comprising titanium or a titanium alloy, nickel or a nickel alloy, MP35N, stainless steel, polysuflone, PEEK, nylon, polypropylene or polyethylene or any flexible or chip-resistant polymer. The hemodialysis and vascular access system may further comprise a first securing device capable of exerting a radially inward force against the first section at the first indentation region. The first securing device may comprise a suture, suture, a twisted wire, a tension clip, a crimp ring, a clamshell or collet assembly, or a compression sleeve. The middle segment may also comprise a mechanical interlock interface capable of joining and separating the first section and the second section of the connector. The middle segment comprises a central lumen access interface. The central lumen access interface may comprise a leak-resistant needle puncture access zone. The central lumen access interface may be subcutaneous or transcutaneous. The hemodialysis and vascular access system may further comprise a connector sleeve with a first end and a second end, and a sleeve lumen therebetween, the tubular sleeve having a first expanded sleeve configuration and a second reduced sleeve configuration, the second reduce sleeve configuration capable of exerting a radially inward bias. The compression sleeve may comprise a material selected from the group comprising silicone, polyurethane, a flexible polymer and a chip-resistant polymer. The hemodialysis and vascular access system may further comprise a strain relief assembly positioned about the first section adjacent to the connector. The strain relief assembly may comprise a wire or polymer coil. The connector may have a first wall thickness measured in the central lumen at a distance of no greater than about 1 mm from the opening of the central lumen adjacent to the first section, and a distance of no greater than about 0.5 mm from the opening of the central lumen adjacent to the first section. The connector may also have a first wall thickness measured at the inflection point between the wall of the central lumen and the opening of the central lumen adjacent to the first section. The first wall thickness is generally within the range of about 0.030 mm to about 0.250 mm, sometimes generally within the range of about 0.075 mm to about 0.200 mm, and occasionally generally within the range of about 0.100 mm to about 0.180 mm.

In one embodiment, a vascular access system is provided, comprising an indwelling catheter having a first end with a first outer diameter and adapted to join a body fluid conduit, a second end adapted to be inserted within a vein at in insertion site, the second end having an outside diameter which is less than an inner diameter of the vein at the insertion site and having at least one opening which is distant from the insertion site such that, in operation, body fluid from the body fluid conduit is capable of flowing through the catheter and returned to the vein through at least one opening and blood also flows through the vein uninterrupted around the outside of the second end, and a lumen extending therethrough, wherein the lumen opening about the first end has a diameter at least about 90% of the first outer diameter.

In another embodiment, the invention comprises a hemodialysis and vascular access system, comprising a first body fluid conduit, a second body fluid conduit, and a needle access site, the needle access site comprising a first end having a first outer diameter and adapted to join the first body fluid conduit, a second end adapted to join the second body fluid conduit, and a lumen between the first end and the second end of the needle access site, the lumen comprising an inflow opening and an outflow opening, and a length, a first edge about the inflow opening, and a second edge about the outflow opening, wherein the inflow opening is at least about 90% of the first outer diameter.

Further features and advantages of the present invention will become apparent to those of skill in the art in view of the disclosure herein, when considered together with the attached drawings and claims.

### BRIEF DESCRIPTION OF THE DRAWINGS

The structure and method of using the invention will be better understood with the following detailed description of embodiments of the invention, along with the accompanying illustrations, in which:

FIG. 1A is a cross-sectional schematic view of one embodiment of the connector. FIGS. 1B and 1C depict the connector edges of the connector in FIG. 1A.

FIG. 2A is an exploded view of one embodiment of the connector system; FIG. 2B is a cross-sectional view of the connector system in FIG. 2A when assembled.

FIG. 3 is a schematic representation of one embodiment of the invention comprising a double-tapered connector.

FIG. 4 is a schematic representation of one embodiment of the invention comprising a compression sleeve.

FIG. 5 is a schematic representation of one embodiment of the invention comprising a suture-secured sleeve.

FIG. 6 is a schematic representation of one embodiment of the invention comprising a clamshell-secured sleeve.

FIG. 7 is a schematic representation of one embodiment of the invention comprising clips for securing the graft and/or catheter to the connector system.

FIG. 8 is a schematic representation of one embodiment of the invention comprising a collet-secured sleeve.

FIG. 9 is a schematic representation of one embodiment of the invention comprising a compression ring-secured sleeve.

FIG. 10 is a schematic representation of one embodiment of the invention comprising barbs on the end of a connector end.

FIG. 11 is a schematic representation of one embodiment of the invention comprising a suture-secured connector system.

FIG. 12 is a schematic representation of one embodiment of the invention comprising a two-part connector.

FIG. 13 is schematic representation of one embodiment of the invention comprising an integrated catheter and connector end.

FIG. 14 is an elevation view of one embodiment comprising a preconnected conduit and connector.

### DETAILED DESCRIPTION OF THE PREFERRED EMBODIMENT

Kanterman hypothesizes that the primary causes of the localized stenosis are due to intimal hyperplasia, compliance mismatch between the graft and the native vein anastomosis and turbulent flow (Kanterman R. Y. et al “Dialysis access grafts: Anatomic location of venous stenosis and results of angioplasty.” Radiology 195: 135-139, 1995, herein incorporated by reference in its entirety). In our work, it was hypothesized that these causes could be circumvented by eliminating the venous anastomosis and instead, using a catheter to discharge the blood directly into the venous system. The device developed by GRAFTcath, Inc. to eliminate the venous anastomosis in the AV shunt has a catheter at the venous end and a synthetic graft anastomosed to the artery in the standard fashion.

Although these devices may be may be constructed as a single-piece, integrated device, a multi-piece device comprising separate components that are later joined together may also be designed. A multi-component device may have several advantages. First, a multi-piece device allows switch-out of one or more components of the device. This allows the tailoring of various device characteristics to the particular anatomy and/or disease state, for instance, by using components of different dimensions. This also reduces the cost of treating patients in several ways. It reduces the amount of inventory of a given device by stocking an inventory range of components, rather than an inventory range of complete devices. Also, if an incorrect device is initially selected for use in a patient, only the incorrect component is discarded, rather than the entire device. Second, separate multiple components of a device may be easier to manufacture compared to an integrated form of the device. Third, it may be easier for a physician to implant separate components of a device and then join them together rather than implanting an integrated device. Fourth, it allows the components to be trimmable as needed to accommodate various patient anatomies. An integrated device may be excessively bulky and can slow the implantation procedure, thereby increasing operating room time and costs as well as increasing the risk of physician error.

The interfaces where separate components are joined or attached, however, are potential sources of turbulent flow along the blood flow path of the device. Sharp indentations or protrusions of the lumen will cause alterations in flow at the interface that may result in hemolysis and clot formation. Such an interface may create an increased risk of creep or separation of joined components over time that can worsen the flow characteristics at the interfaces or even result in loss of flow, respectively. Thus, the connector system used to attach the various components may benefit from one or more design features that maintain smooth flow between components through the interface and also resist creep or separation of the joined components. Such a connector system may be used with AV grafts, peripherally inserted central catheters (PICC), implantable infusion catheters with and without fluid reservoirs, implantable infusion pumps, left ventricular assist devices, and any other device where providing laminar flow between two body fluid conduits may be beneficial. For example, such a connector may be used to join an arterial graft and a venous catheter as described by Squitieri in U.S. Pat. No. 6,102,884 and U.S. Pat. No. 6,582,409, and by Porter in U.S. Provisional Application No. 60/509,428, herein incorporated by reference in their entirety. In addition to joining tubular conduits, the connector may also be used to join conduit or reservoir containing devices such as needle access ports as described by Porter in U.S. Provisional Application No. 60/605,681, herein incorporated by reference in their entirety. The connectors may also be integrated with such conduit or reservoir containing devices.

In one embodiment of the invention, a connection system for attaching a catheter to a graft in an AV hemodialysis shunt is provided. The connection system may comprise a biocompatible and/or hemocompatible material. The connection system may also provide for the attaching of a graft and a catheter having different internal and/or outer diameters. In some embodiments of the invention, the connection system provides a lumen with a smooth fluid path from one end of the connection system to the other. The smooth fluid path may reduce the risk of clot formation and hemolysis of red blood cells. The connector system may also have a securing system for resisting disconnection of the joined components. An anti-kink system may also be provided to resist occlusion along portions of the catheter and/or graft. An anti-kink system may be advantageous for an AV graft comprising PTFE or a catheter which is made from silicone or polyurethane that may be prone to bending and/or twisting. It may also be advantageous to preconnect one element to the connector before the start of surgery with then makes the procedure easier to perform in the operating room and it may also reduce the chance of error.

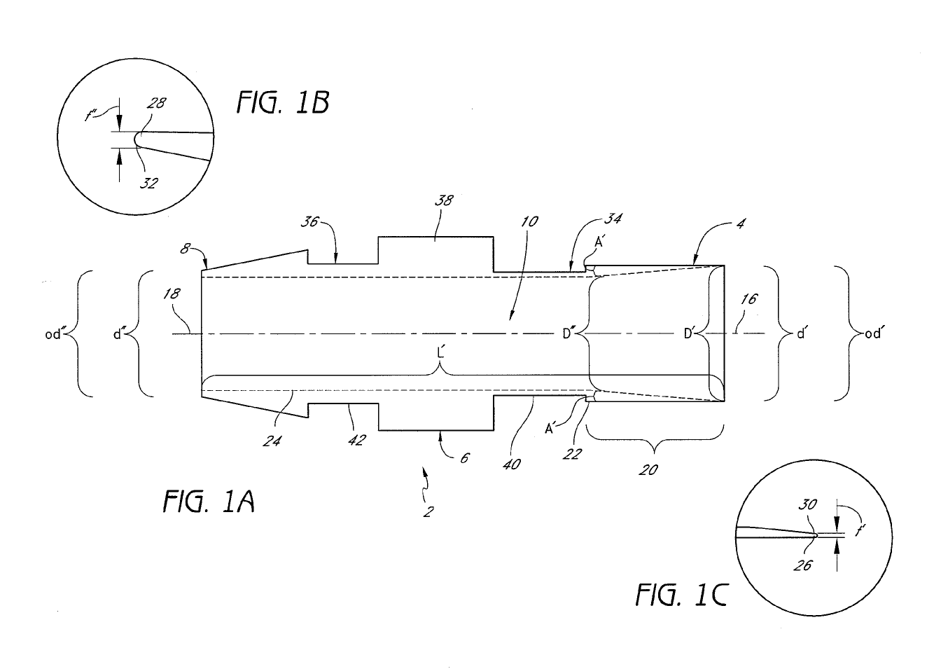

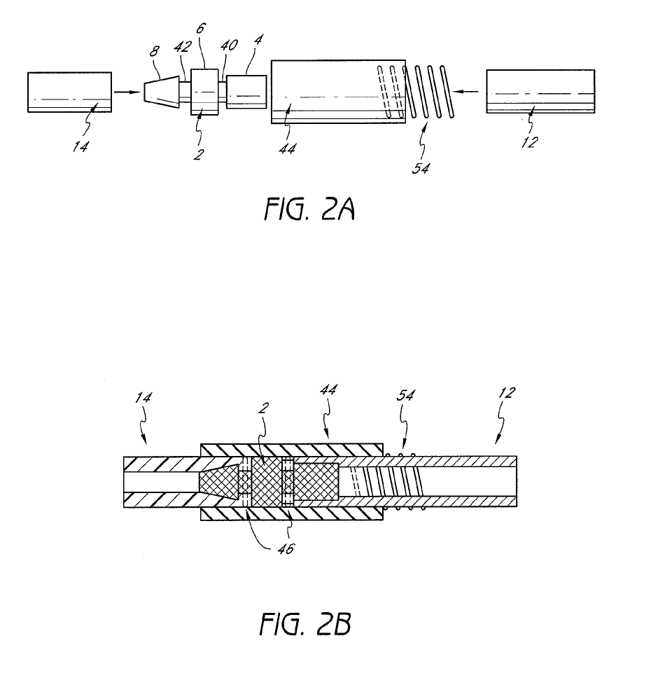

FIG. 1 depicts one embodiment of the invention. The invention comprises a connector 2 having a first end 4 for connecting to a first fluid conduit, a middle portion 6 and a second end 8 for connecting to a second fluid conduit, and a lumen 10 from the first end to the second end. Referring to FIG. 2A, the first fluid conduit 12 is typically a hemodialysis graft component while the second fluid conduit 14 is typically a catheter, but other combinations may also be used, such as graft/graft, catheter/graft or catheter/catheter. Other combinations may also be useful in performing bypass grafts for peripheral vascular disease and liver cirrhosis, and for connecting blood pumps or cardiopulmonary bypass machines. Multiple conduits may also be joined in a serial fashion. The invention disclosed is also applicable to Y-connectors or other branching connectors. The connector may be designed with fluid flow in a direction from the first conduit to the second conduit. This direction of fluid flow may also be defined from upstream to downstream, or from proximal to distal. In other embodiments, the connector may be configured without a particular fluid flow direction.

Where the connector is used to join conduits having generally similar inside diameters, the lumen diameter of the connector may be generally constant from the proximal portion of the first end to the distal portion of the second end. More typically, however, the conduits have different inner diameters, where the first fluid conduit has a greater diameter than the second fluid conduit. Referring back to FIG. 1A, in such circumstances, the most proximal portion 16 of the lumen 10 generally has a larger diameter d′ and the most distal portion 18 of lumen generally has a smaller diameter d″. A smooth transition between the larger diameter d′ and the smaller diameter d″ is provided to reduce turbulent or non-laminar blood flow and hemolysis that may result from abrupt changes in diameter. The change in diameter may be any non-abrupt transition, and may be linear or non-linear. The transition in lumen diameter occurs in a transition zone 20 occupying a portion or the entire length of the lumen 10, but preferably at least about 20% of the lumen length L′, sometimes at least about 25% of the lumen length L′, other times at least about 50% of the lumen length and occasionally over at least about 90% to about 100% of lumen length L′. In some embodiments, the tapering or diameter change of the lumen 10 occurs at no more than about a 30 degree angle as measured on a longitudinal cross section of the connector by the angle A′ between the lumen wall 22 and a line parallel to the longitudinal axis of the lumen and intersecting the lumen wall at the most proximal portion 16. In other embodiments, the diameter change of the lumen 10 occurs at no more than about a 20 degree angle. In some embodiments, the tapering occurs at no greater than about a 10 degree angle, or no greater than about a 5 degree angle. In still other embodiments, the diameter of the connector changes as a percentage of the largest lumen diameter per unit percentage of lumen length. For example, in one embodiment, the diameter decreases by no more than about 3% of the largest lumen diameter per 1% of the lumen length. In other embodiments, the diameter decreases by no more than about 2% of the largest lumen diameter per 1% of the lumen length, and in still other embodiments, the diameter decreases by no more than about 1% or 0.5% of the diameter per 1% of the lumen length. One skilled in the art can select the length of the transition zone based upon the total length of the lumen and/or the amount of diameter change required.

In other embodiments, the first fluid conduit 12 may have a smaller diameter than the second fluid conduit 14 and the connector 2 may be configured so that the most proximal portion 16 of the lumen 10 generally has a smaller diameter and the most distal portion 18 of lumen 10 generally has a larger diameter.

In one embodiment, the transition zone 20 of the connector 2 where the lumen diameter transitions from the larger diameter D′ to the smaller diameter D″ is preferably located at the most proximal portion 16 of the connector and extends distally to at least to the distal portion 22 of the first end 4. The transition zone 20 may also begin at the distal portion 22 of the first end 4, the middle portion 6, or the proximal portion 24 of the second end 8 of the connector 2, and terminate at the middle portion 6, the proximal portion 24 of the second end 8 or the distal portion 18 of the second end 8 of the connector 2, depending on the length of the transition zone 20 desired. FIG. 1A depicts one embodiment with a transition zone 20 from a larger diameter D′ to a smaller diameter D″ generally within the first end 4 of the connector 2, and a constant diameter d″ within the remaining portions of the lumen 10. As shown in FIG. 1C, a transition zone 20 with a larger diameter D′ located at the most proximal portion 16 of the first end 4 may be advantageous because it allows a smaller thickness t′ of connector material at the leading edge 26 of the connector. The reduced connector wall profile or thickness provides a smaller effective surface area that is perpendicular to the fluid flow from the first conduit 12 to the connector 2, thereby reducing disruption of laminar flow, yet maintains the integrity of the connector 2 by allowing an increased connector material thickness as the internal diameter of the connector lumen tapers.

A connector may also have more than one transition zone. Referring to FIG. 3, in one embodiment, the connector 3 comprises a lumen 5 with a first transition zone 7 and a second transition zone 9. The second transition zone 9 has a third inside diameter 11 that is smaller than its fourth inside diameter 13, thus a transition zone may be configured to go from a smaller diameter to a larger diameter, as well as a larger diameter to a smaller diameter.

FIGS. 1B and 1C depict one embodiment of the reduced thickness of the connector wall t′, t″ at the edges 26, 28 of the connector 2. The reduced connector wall thicknesses t′, t″ allows the lumen 10 of the connector 2 to remain generally flush or nearly flush with the lumens of the conduits joined at each end 4,8. In some embodiments, the connector wall thicknesses are configured to reduce t′ and t″ sufficiently to decrease the flow disturbance in the lumen while having an edge profile shaped in such a way that reduces the risk of cutting the lumens of the tubing or pose a hazard to the surgeon. In one embodiment, the connector wall thicknesses are optimized to reduce t′ and t″ as small as possible to prevent flow disturbance in the lumen while having an edge profile shaped in such a way that it does not cut the lumens of the tubing or pose a hazard to the surgeon. For some embodiments, the thickness of the connector wall t′, t″ may be determined at a measurement point in the lumen about 0.5 mm or 1.0 mm from the lumen opening. The measurement point of the thickness t′, t″ of the connector wall may also be defined at the inflection point 30, 32 where the connector edge 26, 28 joins the linear lumen wall as identified on a longitudinal cross section of the connector 2. Where the connector edges 26, 28 are rounded or smoothed, the inflection points are where the curves of the edges 26, 28 meet the linear lumen wall edges as defined on a longitudinal cross section of the connector 2. In some embodiments of the invention, the connector edges 26, 28 at the ends 4, 8 of the connector 2 generally have a thickness t′, t″ no greater than about 20% of the inner diameter of the lumen d′, d″ at the most proximal portion 16 and most distal portion 18 of the lumen 10, respectively. In some instances, the thickness of at least one connector edge t′, t″ is less than about 10% of the inner diameter d′, d″ of the lumen 10 at one connector edge, respectively, and in still other circumstances, the thickness t′, t″ is preferably less than about 5% or about 3% of the inner diameter d′, d″ of the lumen 10, respectively. The connector wall thickness t′, t″ may also be defined relative to the outer diameter od′, od″ of the connector 2 at the same measurement point. Thus, the connector wall thickness t′, t″ may be no greater than about 20% of the outer diameter od′, od″ of the connector 2, respectively, and in some instances no greater than about 10% of the outer diameter od′, od″ of the connector 2, respectively, and preferably less than about 5% or 3% of the outer diameter od′, od″ of the connector 2 at the measurement point, respectively.

As depicted in FIG. 1C, in another embodiment, the thickness t′ of the edge 26 of the first end 4 at the selected measurement point is generally within the range of about 0.030 mm to about 0.250 mm, sometimes within the range of about 0.075 mm to about 0.200 mm, and occasionally about within the range of about 0.100 mm to about 0.180 mm. As illustrated in FIG. 1B, in another embodiment, the thickness of the trailing edge 28 of the second end 8 is generally within the range of about 0.030 mm to about 0.400 mm, sometimes within the range of about 0.125 mm to about 0.300 mm, and occasionally within the range of about 0.175 mm to about 0.250 mm.

To further reduce flow turbulence or non-laminar flow and prevent damage to the surface of the inner surface of the conduits at one or more edges 26, 28 of the connector 2, the first end 4 and/or second end 8 of the connector 2 may be advantageously rounded or smoothed. Rounded edges may also decrease the risk of trauma to the conduits 12, 14 during insertion of the connector 2 into the conduits 12, 14. As shown in FIGS. 1B and 1C, the rounded edges may have a generally semi-circular cross-section, but the edges may also have a cross-section with a generally partial elliptical profile or polygonal profile. For embodiments having a semi-circular cross-sectional edge, the radius of the edge 26, 28 is generally about half of the thickness of the edge at the selected measurement point. Typically, the edge radius is within the range of about 0.025 mm to about 0.200 mm, and sometimes within the range of about 0.025 mm to about 0.125 mm, or occasionally within the range of about 0.075 mm to about 0.100 mm. The rounding or smoothing of the connector edge may be performed using electropolishing, mechanical polishing, or a chemical etchant such as hydrofluoric acid.

The outer diameter od′ of the first end 4 of the connector 2 may be generally constant or it may taper from distal to proximal. In some circumstances, a first end 4 with a generally constant outer diameter may be preferable because the generally constant outer diameter reduces the deformation of the first conduit 12 at the junction of the connector edge 26 and the first conduit 12. The reduced deformation may preserve the structural integrity of the first conduit 12 when joined to the connector 2. It may also reduce the inward deformation that may occur at the junction of the connector edge 26 and the first conduit wall, which can provide a smoother fluid path transition from first conduit 12 to the connector 2. A tapered end, however, may facilitate insertion of the connector 2 into the lumen of the first conduit 12 while providing resistance to separation between the conduit 12 and connector 2.

The outer diameter od″ of the second end 8 of the connector 2 may also be generally constant or have a taper to facilitate insertion into the second conduit 14. In some embodiments, a tapered outer diameter of the connector 2 may be preferred because the effect on flow dynamics, if any, from the lumen 10 of the connector 2 to the larger lumen of the second conduit 14 may not be significant. A taper at the second end 8 of the connector 2 may facilitate insertion of the second conduit 14 with little or no increase in flow turbulence or non-laminar flow. The configuration of one or both connector ends 4, 8 may be the same or different, and may be selected by one skilled in the art depending upon the flow direction, desired flow characteristics, conduit materials and characteristics, and other factors.

The middle portion 6 of the connector 2 has a proximal end 34 adjacent to the first end 4 of the connector 2, a distal end 36 adjacent to the second end 8 of the connector 2, and contains a segment of lumen 10. In one embodiment of the invention, the middle portion 6 has a radially outwardly extending annular flange 38 along at least one portion of its outer diameter that limits the insertion of the first end 4 and second end 8 into their respective conduits 12, 14. The insertion limit may prevent overinsertion of the connector 2 into the conduit, resulting in possible loss of the connector and/or damage to the conduit.

In some embodiments, the middle portion 6 of the connector 2 comprises one or more regions with indentations or a reduced outer diameter 40, 42 with respect to the adjacent outer diameters of the first end 4 and/or second end 8 of the connector 2. Preferably, the connector 2 has a first reduced outer diameter region 40 such as an annular recess adjacent to the first end 4 of the connector 2 and a second reduced outer diameter region 42 such as an annular recess adjacent to the second end 8 of the connector 2, but this is not required. The two regions 40, 42 need not be configured similarly. The regions 40, 42 on the middle portion 6 of the connector 2 allow conduits 12, 14 inserted over the first end 4 and/or second end 8 of the connector 2 to be secured to the connector 2 by placing a radially inward force on the conduits 12, 14 that can partially deform the conduits 12, 14 radially inward and increase resistance to separation from the connector 2 through a friction fit and/or mechanical interfit by abutting against the larger diameter of the first end 4 and/or second end 8 of the connector 2. The indentation or reduced outer diameter regions 40, 42 may involve only a portion of the circumference of the connector 2, but typically will involve the entire circumference of the connector 2. Structures for securing the conduits 12, 14 onto the connector 2 are described in further detail below.

In one embodiment, the connector 2 has a length of about 10 mm to about 50 mm, and preferably about 15 mm to about 30 mm and more preferably about 20 mm to about 25 mm. The connector may comprise any of a variety of biocompatible materials, such as titanium or a titanium alloy, nickel or a nickel alloy, MP35N, stainless steel, polysuflone, PEEK, nylon, polypropylene or polyethylene or any flexible or chip-resistant polymer. All or a portion of the outer and/or inner surface of a metallic connector may be passivated or anodized. All or a portion of the outer and/or inner surface of the connector may be coated or insert molded with silicone or other hemocompatible material to provide a lubricious characteristic or to augment other properties of the connector, such as corrosiveness and/or clot formation. The connector may further comprise a drug eluting surface capable of eluting a therapeutic agent that can reduce the risk of infection, clot formation or affect tissue growth about the connector 2.

FIGS. 2A and 2B depict one embodiment of the invention comprising a first conduit 12, second conduit 14, a connector 2 and a connector sleeve 44. The connector sleeve 44 comprises a tubular structure capable of fitting over the connector 2 and at least one and preferably both conduits 12, 14 joined to the connector 2. The connector sleeve 44 may be capable of applying a radially inward compressive force onto the connector 2 and joined conduits 12, 14. The compressive force may further depress portions of the conduits 12, 14 into the reduced outer diameter regions 40, 42 of the connector 2 and further secure the conduits 12, 14 onto the connector 2. In some embodiments where the connector sleeve 44 is positioned to extend beyond the first end 4 and/or second end 8 of the connector 2, the compressive force may impart a slight radially inward deformation of the joined conduits 12, 14 relative to the connector edges 26, 28 that may reduce the difference, if any, between the lumen diameter of the conduit and the lumen diameters d′, d″ of the connector ends 4, 8 to which the conduits 12, 14 are joined. The connector sleeve 44 may also reduce exposure of any crevices or spaces along the outer surfaces of the connector 2 and thereby eliminate infection risk posed by such areas. Although a single sleeve 44 is depicted in FIGS. 2A and 2B, separate sleeves to cover and/or compress each conduit may also be used.

In one embodiment, the connector sleeve 44 comprises silicone, polyurethane or other polymer in its unexpanded state, has an average inner diameter less than that of the largest outer diameter and/or average outer diameter of the connector 2. The connector sleeve 44 is radially expanded as it is placed over the connector 2 and joined conduits 12, 14, thereby imparting a radially inward compression force.

In another embodiment, the connector sleeve 44 comprises a polymer that may be UV or heat shrunk onto the connector 2. UV and heat shrink polymers include but are not limited to PTFE, FEP, PFA, PET, and PTFE/FEP. In still other embodiments, the connector sleeve 44 may be adhered to the connector 2 and/or conduits 12, 14 with cyanoacrylate, a curable glue, or other adhesive. In still another embodiment, the connector sleeve 44 comprises a tubular lattice structure similar to a stent that is crimped onto the connector system. The stent may also comprise a shape memory material such as Nitinol that is capable of expanding with increased temperature and reducing in diameter with cooling to apply a radially inward force to the sleeve 44 or connector 2.

Securing structures or devices may be applied to the conduits to secure the conduits to the connector. These securing devices 46 may be applied directly to the outer surface of the conduits 12, 14, as shown in FIG. 2B, or they may be applied indirectly on the outer surface of the connector sleeve 44, or both. Application of one or more securing devices 46 onto the connector sleeve 44 may prevent or resist migration of the sleeve 44 with respect to the connector 2. The securing structures are described in greater detail below.

The radially inwardly facing surface of the connector sleeve 44 may also comprise at least one inner ring, indentation or other structure that is complementary to a corresponding structure on the outside surface of the connector and/or conduits that can facilitate positioning and/or securing of the sleeve 44 onto the connector 2. For example, the sleeve 44 may have a radially inwardly extending ring or thread that is complementary to a circumferential indentation area 40, 42 on the connector 2. The inner ring of the sleeve 44 may be segmented and complementary to a series of circumferential indentations on the connector to facilitate rotational alignment of the sleeve and connector in addition to longitudinal alignment.

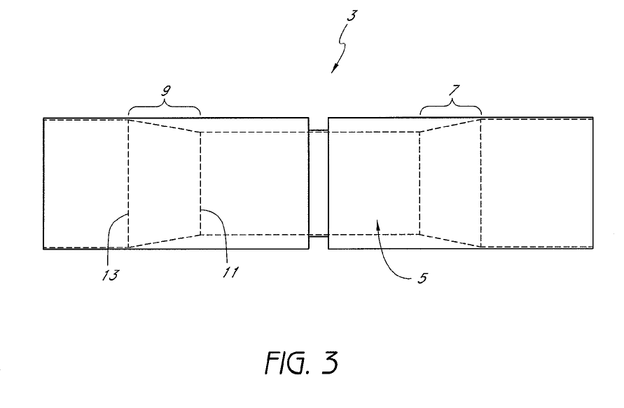

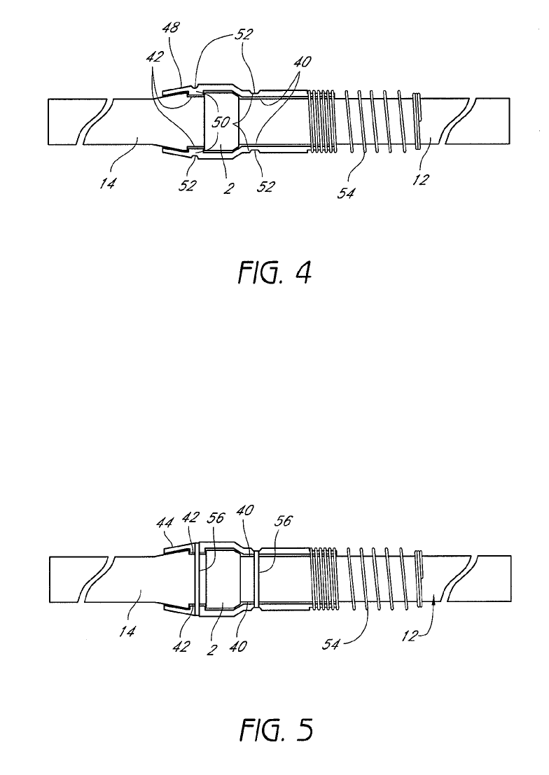

FIG. 4 illustrates one embodiment where the securing device comprises a compression sleeve 48 with radial protrusions 50 on the inner surface of the sleeve 48 capable of exerting radially inward pressure along the indented or reduced diameter portions 40, 42 of the connector 2 and/or conduits 12, 14 when positioned over the connector 2 and joined conduits 12, 14. The compression sleeve 48 may also have indentation points or regions 52 on its outer surface to facilitate use of other securing devices 46 such as clips, rings, sutures or others disclosed elsewhere herein to provide supplemental compression of the compression sleeve 48 onto the connector system.

In some embodiments of the invention, the interior surface of the connector sleeve 44 may have a lubricious coating to facilitate sliding of the sleeve 44 over the connector 2 and/or conduits 12, 14. The sleeve 44 may also comprise a porous material to facilitate tissue ingrowth and fixation of the connector system position within the body. Fixation of the connector system position may be advantageous when attempting puncture or obtain access to the joined conduits/grafts by preventing rolling or lateral displacement of the conduits caused by a puncturing force.

As shown in FIGS. 2A and 2B, the invention may further comprise a strain relief structure 54 to resist kinking of one or more conduits or grafts attached to the connector 2. This may be advantageous for conduits or grafts that comprise PTFE or other flexible materials and may prevent occlusion of the conduit or graft. The strain relief structure 54 typically comprises a flexible spiral or coil that extends from an end of the connector system and onto the outer surface of or within the wall of the conduit/graft. The strain relief structure may comprise a biocompatible metal or plastic. Other strain relief structures that may be used include a tubular or trumpet-shaped strain relief structure. The strain relief structure may be a separate structure from the connector 2 and/or connector sleeve 44, or may be embedded or integrated with the connector 2 or sleeve 44. FIG. 2A is a schematic of a connector system with a connector sleeve 44 and a separate strain relief structure 54. When all components are joined together as in FIG. 2B, the first conduit/graft 12 is inserted into the strain relief structure 54 and over the first end 4 of the connector 2. The second conduit 14 is inserted over the second end 8 of the connector 2. Both the first conduit 12 and second conduit 14 are secured to the connector 2 using securing devices. A connector sleeve 44 is located over a portion of the strain relief structure 54, first conduit 12, central flange 38, second conduit 14 and the securing structures 46 securing the first 12 and second conduits 14. A portion of the strain relief structure 54 is layered between connector sleeve 44 and first conduit 12 and is maintained at its position by radial compression from the connector sleeve and/or radial compression from the strain relief structure 54 onto the conduit 12.

Any of a variety of securing devices may be used to secure the conduits and/or connector sleeve 44 to the connector 2. FIG. 5 is a schematic view of one embodiment of the invention utilizing sutures 56 or wires to secure the conduits 12, 14 and connector sleeve 44 to the connector 2. The connector sleeve 44 is shown in cross-section to illustrate the interaction of the suture/wire 56, conduits 12, 14 and sleeve 44 with the reduced diameter portions 40, 42 of the connector 2. In one embodiment, one or more securing devices comprise non-absorbable sutures well known in the art, and are tied around the connector sleeve 44 and conduits 12, 14 about the reduced diameter portions 40, 42 of the connector 2. In other embodiments, the securing device comprises a wire that is wound around the connector system and twisted several times to tighten the wire. FIG. 5 also depicts one embodiment of the strain relief assembly 54 that is positioned concentrically around the outer surface of the connector sleeve 44.

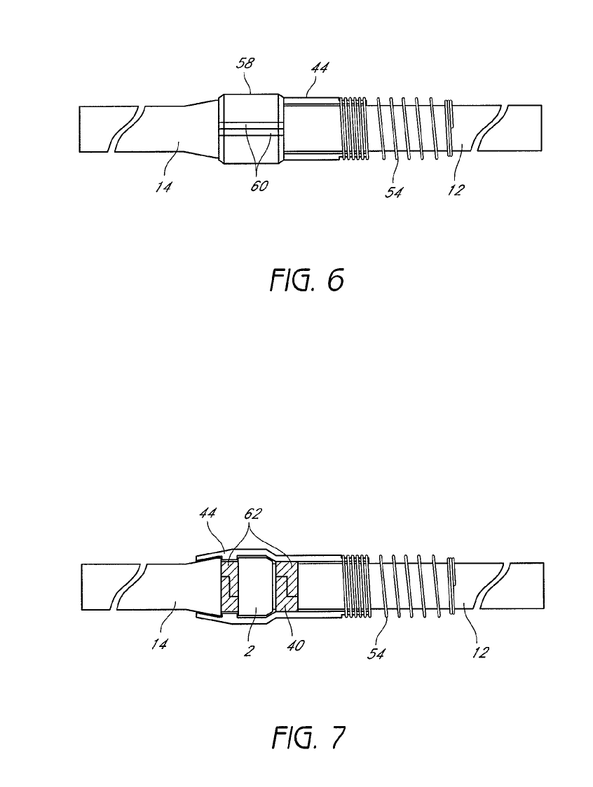

FIG. 6 depicts another embodiment of the invention where the securing device comprises a clamshell assembly 58 configured to clamp around a portion of the connector 2. The clamshell assembly 58 may have one or more radially inwardly extending protrusions that interface with the reduced diameter portions 40, 42 or indentation points on the connector 2 that secure the conduits 12, 14 and sleeve 44 onto the connector 2. The clamshell assembly 58 may be configured to secure the conduits 12, 14 at one or both ends 4, 8 of the connector 2. A two-end clamshell assembly 58 is depicted in FIG. 5. The clamshell assembly 58 is generally C-shaped comprise a pair of complementary connecting structures 60 that can be joined to close the C-shape and form a tubular structure around the connector 2. The connecting structures 60 may be any of a variety of snap fits or other mechanical interfits.

FIG. 7 is another embodiment where the securing devices comprise tension clips 62. The tension clips 62 are deformable C-shaped devices adapted for placement about the indentation points or regions 40, 42 of a connector 2 and are capable of exerting radially inward force as the arms of the tension clips 62 are separated. The tension clips 62 may have a rectangular, square, circular, elliptical, triangular or other polygonal cross-sectional shape. The width of the clip 62 for each end 4, 8 of the connector 2 may be the same or different. The cross-sectional shape and/or width of each clip 62 may be the same or different along the circumference of the clip. The cross-sectional shape and width may be selected based upon the particular material and characteristics of the conduit attached at that particular connector end. For example, a conduit or graft comprising PTFE may be more prone to damage with a relatively high securing force and may benefit from a tension clip 62 that exerts less force per surface area but maintains sufficient securing force through a wider clip with increased surface area. A catheter-type conduit, however, may comprise a more durable material than PTFE and can withstand higher radial compression force from a thinner clip that has an inverted triangle cross-sectional shape that is capable of applying a higher compression force at the bottom tip of the triangle, for example. In another embodiment, the tension clip may also be crimped to further increase the radial force acting on the connector and to secure the conduits. In still another embodiment, the securing device comprises a crimp ring that may lack inherent tension and is crimped onto the connector system to secure the joined conduits to the connector.

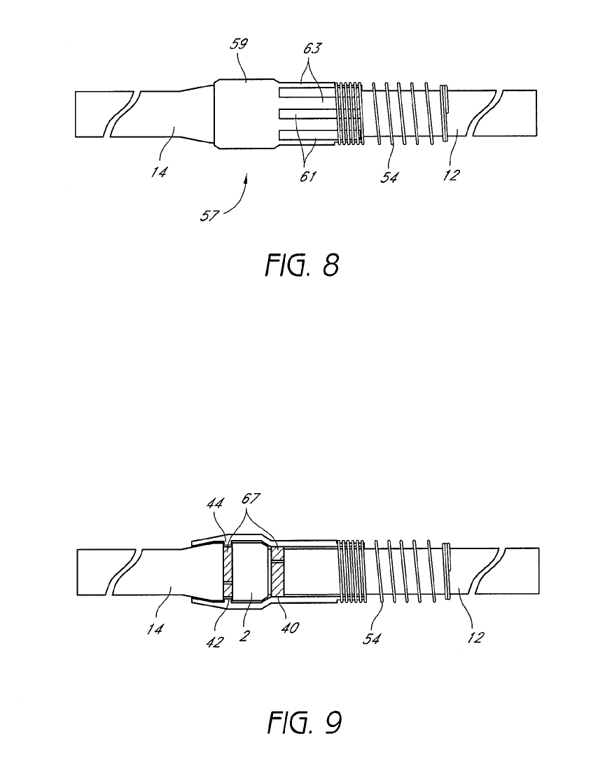

FIG. 8 illustrates another embodiment of the invention comprising a collet securing device. In one embodiment, the collet 57 comprises a tubular assembly 59 with a series of radially-spaced longitudinal slits 61 between prongs 63 of the tubular assembly 59. After the conduits 12, 14 are attached to the connector 2, the collet 57 is slipped over the joined connector system. The prongs 63 may or may not have a radially inward bias capable of applying radially inward force against the connector sleeve 44 and/or conduits 12, 14. The prongs 63 of the collet 57 may be crimped to increase the radially inward force exerted by the collet 57. A strain relief assembly 54 may be placed around the prongs 63 of the collet 59 with sufficient radially inward force to at least secure the strain relief assembly 54 and may or may not exert radially inward force to further secure the sleeve 54 or conduits 12, 14. The collet may be configured to secure one or both of the conduits 12, 14.

FIG. 9 illustrates another embodiment of the invention where the securing device comprises a crimp or compression ring/collar 65. The compression ring/collar 65 is slipped over one or both conduits 12, 14 joined to the connector 2 and then collapsed with a crimp tool onto the surface of the conduits 12, 14. The compression ring/collar 67 may also be slipped over the connector sleeve 44 overlying the joined conduits and connector. The compression ring/collar 67 may then be crimped to secure the connector sleeve 44 in addition to the joined conduits 12, 14. As depicted in FIG. 9, the connector sleeve 44 may also be positioned onto the connector system after crimping of the compression rings or collars 67. The compression ring/collar 67 may have any of a variety of cross sectional shapes, including circular, oval, square, rectangular, triangular or other polygonal shape. The cross sectional shape of the compression ring/collar may be complementary to the corresponding indentation regions 40, 42 of the connector 2.

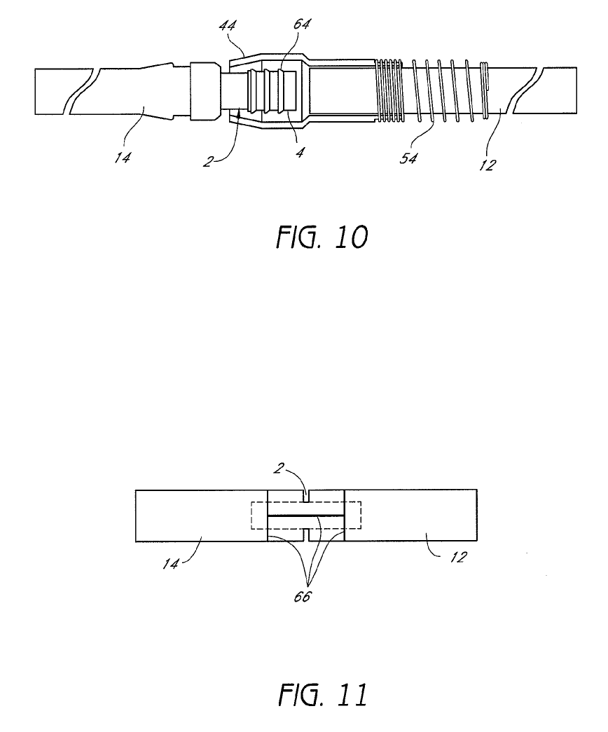

FIG. 10 depicts one embodiment of the invention comprising one or more barb-like protrusions 64 along the outer surface of at least one end 4 of the connector 2. The barb-like protrusions 64 may completely encircle the end 4 of the connector 2, as shown in FIG. 10, or partially encircle the connector end. The barb-like protrusions 64 include a ramped surface which inclines radially outwardly from the base of the protrusion to the tip of the protrusion in a direction away from the connector end 4. This orientation allows relative ease of insertion of the conduit 12 over the connector 2 but resists separation of the conduit 12 from the connector end 4. The barb-like protrusions 64 in FIG. 10 are located at the first or inflow end 4 of the connector 2 having a constant outside diameter, but may also be located on the second or outflow end 8 of a connector 2, or on a connector end with a tapering outside diameter.

In one embodiment, shown in FIG. 11, the invention comprises a connector 2 without a central flange. This embodiment of the invention allows the ends of the two conduits 12, 14 to come in contact with each other and to encase the connector 2 completely. This embodiment minimizes surface protrusions along the AV graft. To secure the two conduits 12, 14, sutures 66 may be used to tie each conduit 12, 14 directly to the other conduit. Other securing devices, such as tension clips 62 or a clamshell/collet assembly 58, may be attached around the conduits 12, 14 about the connector 2, but these devices may increase the surface profile of the AV graft.

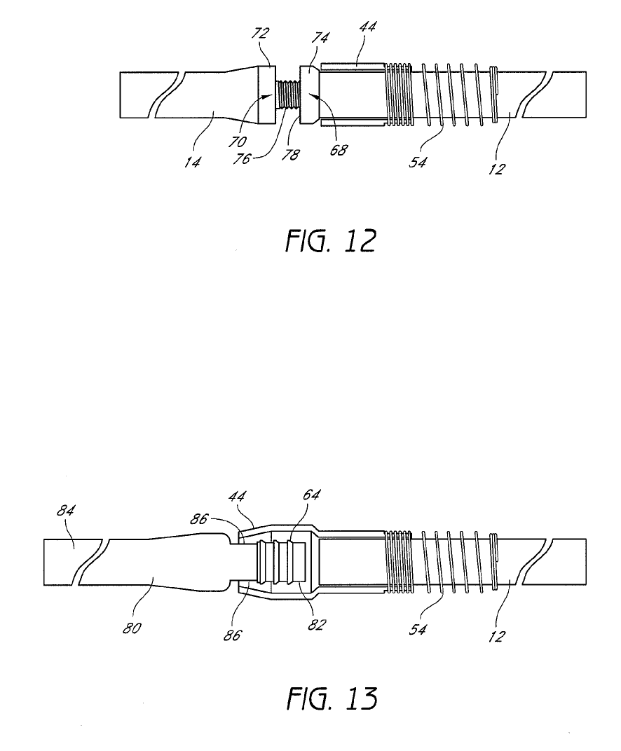

FIG. 12 illustrates an embodiment of the invention comprising a two-component 68, 70 connector. The first component 68 and second component 70 of the connector each comprises a first end and a second end 72, 74 with a lumen therethrough. The first ends are adapted to receive a catheter or graft conduit. Each second end 72, 74 comprises a securing region for attaching a securing device to each component of the connector to secure the conduit to the connector component. Each second end also comprises a complementary portion 76, 78 of a mechanical interlock interface which is capable of releasably or permanently joining the two components 68, 70 of the connector. The mechanical interlock interface may comprise a male/female luer or other threaded interface, a flare or compression fit, or any other sealable mechanical interfit known in the art.

In one embodiment of the invention, the connector system comprises a catheter 80 integrated with a connector-like end 82. FIG. 13 illustrates a catheter 80 comprising a first end 82 adapted for receiving a conduit or graft, a second end 84 configured for insertion into a vein, and a lumen from the first end to the second end. The second end 84 of the catheter 80 comprises a rounded connector edge and/or reduced catheter wall thickness at the selected measuring point as previously described. The second end 84 of the catheter 80 may further comprise one or more indentation points or regions 86 for securing the conduit or graft to the first end 82 of the catheter 80 with a securing device. A connector sleeve 44 may be placed over the second end of the catheter and graft to secure the graft to the catheter and/or to reduce exposure of the catheter/graft joint to the body.

In another embodiment of the invention, an AV shunt comprising a first body fluid segment, a second body fluid segment and a connector is provided. The first body fluid segment is configured for attachment to an artery and the second body fluid segment is adapted for insertion into a vein. The first body fluid segment may comprise a synthetic vascular graft. The synthetic vascular graft comprises a porous structure made from materials such as PTFE, polyurethane or silicone. In some embodiments of the invention, access to the AV shunt may be obtained by direct needle puncture of the vascular graft. The synthetic vascular graft may also comprise a biological material derived from humans or animals. Some embodiments of the vascular graft may be using needles or other access device after a maturation period, while other embodiments of the vascular graft may be used immediately following implantation of the graft.

The second body fluid segment may comprise a catheter or other conduit that is adapted to transport blood or other body fluid into the venous system. The second body fluid segment may have a first outer diameter that transitions to a second outer diameter adapted for insertion into a vein. In one embodiment, the second outer diameter may be within the range of about 3 mm to about 10 mm, sometimes within the range of about 4 mm to about 8 mm, and preferably about 5 mm. In some embodiments, the second body fluid segment is designed to be trimmable at the point of use to facilitate further customization of the device to a particular patient. The second body fluid segment may also have an embedded or external spiral support to provide kink resistance.

The selection of the inner diameter, outer diameter and length of the two segments may be selected by one skilled in the art, based upon factors including but not limited to the vein into which the second body fluid segment is being inserted into, the length of catheter to be inserted through the vein wall, as well as the desired flow rate and fluid resistance characteristics.

In one embodiment, the invention further comprises a conduit access or needle access site. The needle access site may be on the catheter and/or the graft, involving direct puncture of the catheter and/graft components with a needle. The invention may further comprise a separate needle access site structure attached to the catheter, graft or to both, using one or more connectors. The conduit access site may be subcutaneous or transcutaneous. Access to the conduit is typically obtained by using needle puncture, but other sealable or valved interfaces capable of non-piercing access are known in the art and may also be used.

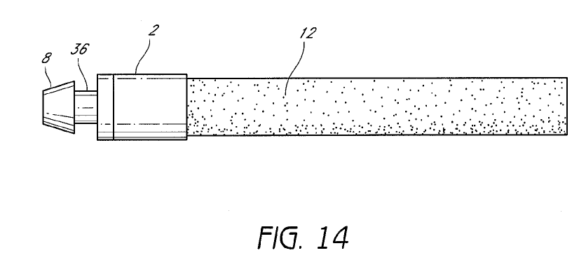

In one embodiment, the invention comprises a method of forming an AV hemodialysis graft. A connector system comprising a graft, a catheter and a connector is provided. The first end of the graft is attached to an artery in the body and the second end of the catheter is inserted into the lumen of a vein. The second end of the graft is attached to the first end of the connector and the first end of the catheter is attached to the second end of the connector. The artery may be the radial artery, ulnar artery, brachial artery, axial artery, femoral artery, popliteal artery, anterior tibial artery, posterior tibial artery, dorsalis pedis artery, hypogastric artery, external iliac artery, thoracic aorta, abdominal aorta, common carotid artery, external carotid artery, internal carotid artery, vertebral arteries, renal artery or any other artery where AV anastomosis is desired. The vein may be a cephalic vein, basilic vein, brachial vein, axillary vein, subclavian vein, a pulmonary vein, an innominate vein, internal mammary vein, azygous vein, a basivertebral vein, an intervertebral vein, external jugular vein, internal jugular vein, a vertebral vein, saphenous vein, popliteal vein, femoral vein, deep femoral vein, external iliac vein, common iliac vein, hypogastric vein, the inferior vena cava, the superior vena cava, renal vein, hepatic vein, portal vein or any other vein or a lymphatic duct in the body. In some embodiments of the invention, the connector may be attached to the graft and/or catheter at the point of manufacture. In some embodiments, the connector may be attached to the graft and/or catheter prior to attaching or inserting the graft and/or catheter to the blood vessel, respectively. FIG. 14 depicts one embodiment of the invention comprising a connector 2 preconnected to a conduit 12.

While this invention has been particularly shown and described with references to embodiments thereof, it will be understood by those skilled in the art that various changes in form and details may be made therein without departing from the scope of the invention. For all of the embodiments described above, the steps of the methods need not be performed sequentially.

What is claimed is:

1. A method for implanting a body fluid conduit, comprising the steps of:
providing a first body fluid conduit, a second body fluid conduit and a connector having a first end, second end, a lumen between the first end and second end, and a first wall thickness at the first end, wherein the first wall thickness is no greater than about 0.250 mm;
attaching the first body fluid conduit to a blood vessel;
inserting the second body fluid conduit into a blood vessel;
connecting the second body fluid conduit to the second end of the connector.

2. A method for implanting a body fluid conduit as in claim 1, wherein the step of connecting the first body fluid conduit to the first end of the connector is performed before the attaching step.

3. A method for implanting a body fluid conduit as in claim 1, wherein the step of connecting the second body fluid conduit to the second end of the connector is performed before the inserting step.

4. A method for implanting a body fluid conduit as in claim 1, further comprising connecting the first body fluid conduit to the first end of the connector.

5. A method for implanting a body fluid conduit, comprising the steps of:
providing a first body fluid conduit, a second body fluid conduit and a connector having a first end, second end, a lumen between the first end and second end, and a first wall thickness at the first end, wherein the first wall thickness is no greater than about 0.250 mm, wherein the first body fluid conduit is preattached to the first end of the connector;
attaching the first body fluid conduit to a blood vessel;
inserting the second body fluid conduit into a blood vessel; and
connecting the second body fluid conduit to the second end of the connector.

6. A method for implanting a body fluid conduit, comprising the steps of:
providing a first body fluid conduit, a second body fluid conduit and a connector having a first end, second end, a lumen between the first end and second end, and a first wall thickness at the first end, wherein the first wall thickness is no greater than about 0.250 mm, wherein the second body fluid conduit is preattached to the second end of the connector;
attaching the first body fluid conduit to a blood vessel;
inserting the second body fluid conduit into a blood vessel; and
connecting the first body fluid conduit to the first end of the connector.
